(19) United States
(12) Patent Application Publication
(10) Pub. No.: US 2010/0204676 A1
(43) Pub. Date: Aug. 12, 2010

(54) METHOD AND APPARATUS FOR CHEMICAL DETECTION AND RELEASE

(76) Inventor: Mario W. CARDULLO, Alexandria, VA (US)

Correspondence Address: MCGUIREWOODS, LLP, 1750 TYSONS BLVD, SUITE 1800, MCLEAN, VA, 22102, US

(21) Appl. No.: 12/646,555

(22) Filed: Dec. 23, 2009

Related U.S. Application Data
(60) Provisional application No. 61/140,399, filed on Dec. 23, 2008.
(60) Provisional application No. 61/140,386, filed on Dec. 23, 2008.

(51) Int. Cl. G01N 33/00; A61M 31/00

(52) U.S. Cl. 604/503; 422/83; 436/86; 436/94; 436/71; 977/742; 977/904

(57) ABSTRACT

A nano-sniffer is provided for detecting chemicals and/or releasing chemicals based on detection of a chemical. The nano-sniffer may be less than about 150 nanometers in size. The nano-sniffer may be a passive, active, or semi-passive nano-sniffer. The nano-sniffer may be distributed to a subjects such as a human or animal or products, for example. The nano-sniffer may include a nano RFID component, including nano antennae that may comprise one or more carbon tubes. The nano-sniffer may include a nano battery. The nano-sniffer may include an environmentally reactive shell that reacts to its immediate environment to affix or adhere to a subject. The nano-sniffer may be constructed for direct or indirect distribution techniques such as by airborne techniques for inhalation, consumption distribution for ingestion, and contact distribution, for example.

### CROSS-REFERENCE TO RELATED APPLICATIONS

This application claims priority under 35 U.S.C. §119(e) to provisional U.S. Patent Application No. 61/140,399 and U.S. Patent Application No. 61/140,386, both filed on Dec. 23, 2008, the disclosures of which are expressly incorporated by reference herein in their entirety.

### BACKGROUND OF THE INVENTION

1. Field of the Invention

The invention is directed generally to a device and method for nano radio frequency identification (RFID) and, more specifically, to an apparatus and method for detection of specific chemicals and targeted chemical release.

2. Related Art

A large number of fields suffer from imprecise detection of critical targets or would benefit from improved sensitivity in detection. Many of these fields, including certain medical disciplines, rely on the detection of chemical markers. In other fields, such as pest control, chemical detection can be used in place of traditional detection methods, such as visual inspection.

In combating targets, it is typically necessary to accurately identify an individual targets for extermination and, then, to exterminate the targets using a physical device (such as, e.g., a trap) or a chemical agent (such as, e.g., poison). Targets have been commonly identified through physical observation. These various methodologies have not been reliable in exterminating pests that are elusive and hide in locations that are inaccessible to certain physical devices, or in locations where it is impractical to use certain chemical agents.

In health care, many systems and methods exist for bio-detection or biometrics based on specific chemical signals. Detection is commonly followed with targeted delivery of drugs. Most of these systems and methods, however, tend to be invasive, frequently cause the patient discomfort or pain, and sometimes lead to infections that are life-threatening.

For example, blood sugar level is typically measured on a blood sample obtained from a patient by using either chemical methods or enzymatic methods to determine the glucose concentration in the blood sample. The blood sample is typically obtained from the patient using intravenous methods or through a finger stick. Then, depending on the glucose concentration detected in the blood sample, drugs may be delivered to the patient intravenously to affect catabolic hormones (such as, e.g., glucagon, growth hormone, catecholamines, thyroxine and somatostatin), which increase blood glucose, or anabolic hormones (insulin), which decrease blood glucose.

Other fields rely on chemical detection as well. For example, law enforcement personnel rely on specially trained dogs to detect illegal narcotics and explosive materials. The training for these dogs is expansive and their availability is limited. In addition, dogs are simply not effective in locating explosives in landmines, and landmines pose serious threat to adults and children throughout much of the world.

Accordingly, there is a need for an apparatus and method for reliably detecting chemical markers and responding with a release of one or more chemicals.

### SUMMARY OF THE INVENTION

The invention meets the foregoing need and provides for a nano-sniffer, which comprises a nano RFID component. The invention also provides a related method suitable for use in applications for chemical identification and/or chemical release. The nano-sniffer may include a nano radio frequency identification (RFID) device and a chemical identifier and/or a chemical container.

Accordingly, in one aspect of the invention, a nano-sniffer device includes a radio frequency (RF) component configured to be responsive to an RF signal, a chemical identifier configured to detect a first chemical, a chemical container configured to release a second chemical, antennae operatively coupled to an RF section to receive the RF signal and to emit a response, and a shell surrounding the RF component, the antennae, the chemical detector, or the chemical container. The nano-sniffer is less than 150 nanometers in at least one dimension, i.e., height, width, or length.

The shell may include a protective covering to protect the nano-sniffer or an environmentally reactive layer. The shell may be constructed to facilitate attaching to, or embedding in, a subject. The nano-sniffer may be distributed by airborne delivery and may be inhaled by a subject. The RF component may respond to an RF signal by backscattering the received signal, and the RF component may respond with data identifying a detected chemical. The nano-sniffer's antennae may include one or more carbon nanotubes. The nano-sniffer may include a micro-circuit to process the received signal and memory connected to the micro-circuit to store chemical information. In addition, the nano-sniffer may include a nano power source. The nano power source may power the RF component. Furthermore, the nano power source may power the RF component at least partially, and the emitted response may be emitted by backscatter. The RF component may be dynamically configurable to be responsive or non-responsive to an RF signal based on a state of the shell.

The chemical identifier may include a spectrometer, a nondispersive infrared sensor, a spectrophotometer, a potentiometric sensor, an optrode, a metal oxide semiconductor, a conducting polymer, a quartz crystal microbalance, a surface acoustic wave sensor, a microwave chemistry sensor, a chemiresistor, an electrolyte-insulator-semiconductor sensor, a metal oxide semiconductor field effect transistor, an electrolyte-oxide-semiconductor field effect transistor, or a chemical field effect transistor. The chemical identifier may include a nanosensor or a biosensor. The chemical identifier may include a micro-electromechanical system, a nano-electromechanical system, or a lab-on-a-chip. The first chemical (i.e. the chemical detected) may be a toxin, a poison, a pheromone, a dye, a hormone, an antigen, a peptide, a protein, a nucleic acid, a carbohydrate, a fatty acid, a signaling molecule, a neurotransmitter, or a biological waste molecule. The second chemical (i.e. the chemical released) may be a toxin, a poison, a pheromone, a dye, a hormone, a drug, a pro-drug, an antibiotic, an anti-viral, a neurotransmitter, a protein, a carbohydrate, a fatty acid, a nucleic acid, a signaling molecule, a micro-electromechanical system, a nano-electromechanical system, a peptide, an aptamer, or a quantum dot.

According to another aspect of the invention, a method is provided for using a nano-sniffer equipped with a radio frequency (RF) component that is configured to be responsive to an RF signal. The nano-sniffer also includes antennae operatively coupled to an RF section to receive the RF signal and to emit a response. The nano-sniffer is less than 150 nanometers in at least one dimension, i.e., height, width, or length. The method for using the nano-sniffer includes storing chemical information within the nano-sniffer and distributing the nano-sniffer for chemical detection and/or chemical release.

The nano-sniffer may be configured to be affixed to a human or animal subject. The nano-sniffer may include a shell surrounding the RF section. The shell may surround additional components. The shell may be an environmentally reactive shell, a shell configured for magnetic adherence, a shell configured for electrostatic adherence, or a shell configured for mechanic adherence. The shell may include a protective layer. Distributing the nano-sniffer may involve airborne distribution or contact distribution. Distributing may alternatively involve ingestion by the subject, inhalation by the subject, or insertion into the subject. The response emitted by the nano-sniffer may include chemical information. The method may also include adhering the nano-sniffer to a subject, a location, or an object. Adhesion may be achieved by an environmentally reactive shell of the nano-sniffer, or it may involve a magnetic adherence technique, an electrostatic adherence technique, or a biological adhesive.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced. In the drawings:

FIG. 1A shows an example of a nano-sniffer comprising a nano RFID component, a chemical identifier, and a chemical container constructed according to an aspect of the invention;

FIGS. 1B and 1C show examples of a chemical identifier vessel and a chemical delivery vessel, respectively, constructed according to another aspect of the invention;

FIG. 2 shows a block diagram of an aspect of a nano RFID component constructed according to principles of the invention;

FIG. 3 shows a block diagram of another aspect of a nano RFID component constructed according to principles of the invention;

FIG. 4 shows a block diagram of another aspect of a nano RFID component constructed according to principles of the invention;

FIG. 5 shows a flow diagram of an exemplary process performed according to principles of the invention and programming a nano-sniffer constructed according to principles of the invention;

FIG. 6 shows a flow diagram showing exemplary process for constructing and distributing a nano-sniffer, according to principles of the invention; and

FIG. 7 shows an example of a process for identifying a chemical and sending a release signal, according to principles of the invention.

### DETAILED DESCRIPTION OF THE INVENTION

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

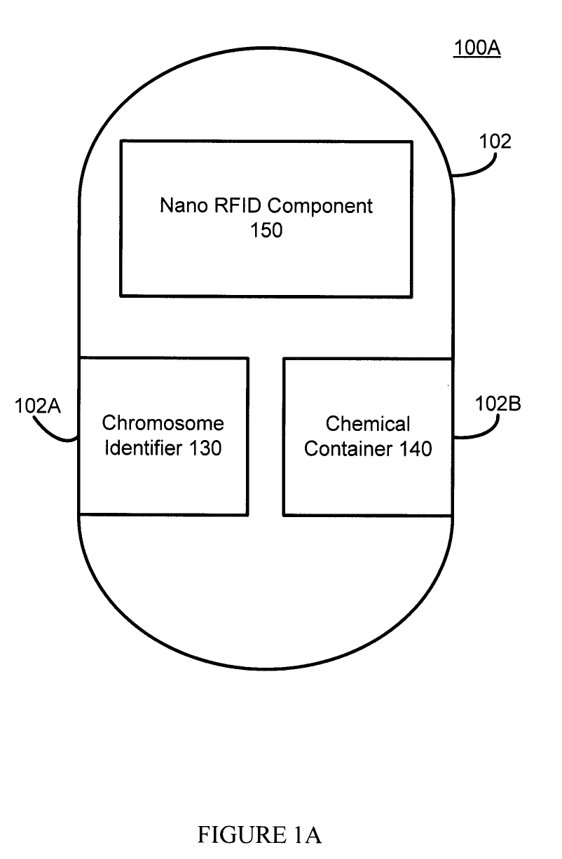

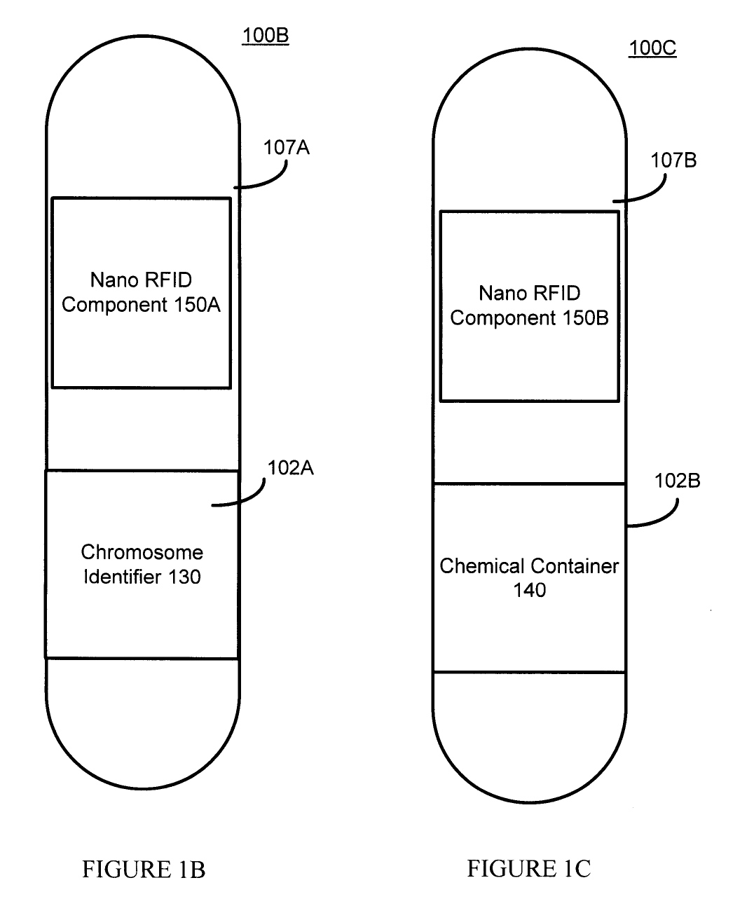

FIG. 1A shows an example of a nano-sniffer 100A including a nano RFID component 150, a chemical identifier 130, and a chemical container 140, all of which may be enclosed or contained in a shell 102, which may provide an adhering or attaching property as discussed more fully below. The nano-sniffer 100A may be delivered to a subject (such as, e.g., a person, an animal, a plant, a micro-organism, or the like) intravenously, via airborne dissemination, through ingestion, or through contact distribution (perhaps by aerosol or a mist, for example).

The nano RFID component 150 may include dimensions of about 150 nanometers or less, in any one, or all of its width (x-dimension), height (y-dimension) and/or length (z-dimension). In some embodiments, the RFID component 150 may include semiconductors as small as 90-nm, perhaps with some chips configured and provided at the 65-nm, 45-nm and/or 30-nm size level, in view of the current cutting edge state-of-the-art in nano-fabrication. The technology for the included electrical circuitry may include CMOS or related technology for low power consumption. The nano RFID component 150 constructed by nanotechnology techniques provides advantages over the currently available RFID devices such as permitting the nano RFID component 150, or the nano-sniffer containing the nano RFID component 150 to be distributed by intravenous delivery, airborne, ingestion, or contact distribution (perhaps by aerosol or a mist, for example), or constructed to react to a specific environmental factor for embedding/affixing to a surface or specific type of material (e.g., an organic material). The invention provides for dynamic distribution and delivery of the nano-sniffer, including the nano RFID component 150, the chemical identifier 130, and/or the chemical container 140 to targeted subjects. Furthermore, the invention provides for effective target identification and/or delivery of chemicals in response to identification of a particular chemical.

The nano RFID component 150 may be configured to receive a chemical detection signal, which may include a chemical identification signal and a chemical concentration signal, from the chemical identifier 130. The nano RFID component 150 may be further configured to send a chemical release signal to the chemical container 140 to release the chemical contained therein based on the received chemical detection signal.

Further, the nano RFID component 150 may be further configured to send the chemical detection signal to an external monitoring device (such as, for example, a RFID reader device) to display information about the detected chemical, including, for example, chemical type, chemical concentration, chemical identification, a date, a time, a location of the RFID component 150, or the like, on a display (not shown). The external monitoring device may include, but is not limited to, for example, an electronic device configured to accept data, perform prescribed mathematical and logical operations at high speed, and output the results of these operations. The external monitoring device may include a computer, such as, for example, but not limited to, a personal computer, a laptop computer, a palmtop computer, a notebook computer, a desktop computer, a workstation, netbook, mobile station, user equipment, or the like. The external monitoring device may further include a reader, a transponder, or the like. Further, the external monitoring device may be strategically located in private or public locations, including but not limited to, for example, airports, bus-terminals, parks, buildings, homes, rooms, offices, lobbies, malls, streets, walkways, or the like.

The nano-sniffer, including the nano RFID component 150, may be passive, thereby avoiding any need for an internal power supply. In this case, an electrical current may be induced in an internal antenna by an incoming RF excitation signal to provide adequate power to run the CMOS integrated circuit(s) in the nano-sniffer, including the RFID component 150, including powering up, generating and sending an RF transmission signal, and receiving an RF reception signal. The RF transmission signal may be sent by backscattering a carrier wave from the external monitoring device (not shown). The RF transmission signal may include, for example, a chemical detection signal, a chemical identification signal (such as, e.g., an individual identification (ID) number that identifies a particular chemical that has been detected), a chemical release signal, or the like. The antenna may be configured to both collect power from the incoming RF excitation signal and also to transmit the outbound RF transmission signal. Further, the CMOS integrated circuit(s) may contain a non-volatile, read-only, or rewritable EEPROM for storing data.

Alternatively, the nano-sniffer, including the nano RFID component 150, may be a semi-passive device which includes a power source, but the power source is used only to power the micro-circuitry and does not power transmission of the RF transmission signal. In this case, transmission of the RF transmission signal may be powered by the backscattering of the RF excitation signal energy from the external monitoring device.

Further, the nano-sniffer, including the nano RFID component 150, may be an active RFID device which includes a power source that provides power for all of the functions of the micro-circuitry and signal transmission and reception.

The nano RFID component 150 may contain at least two parts. The nano RFID component 150 may include an integrated circuit for storing and processing information, modulating and demodulating a radio frequency (RF) signal, and other specialized functions. The nano RFID component 150 may further include an antenna for receiving and transmitting the signal.

The chemical identifier 130 may include any sensor known to one skilled in the art. By way of non-limiting examples, the chemical identifier may be a spectrometer, a nondispersive infrared sensor, a spectrophotometer, a potentiometric sensor, an optrode, a metal oxide semiconductor (MOS), a conducting polymer (CP), a quartz crystal microbalance, a surface acoustic wave (SAW) sensor, a microwave chemistry sensor, a chemiresistor, an electrolyte-insulator-semiconductor (EIS) sensor, a metal oxide semiconductor field effect transistor (MOSFET), an electrolyte-oxide-semiconductor field effect transistor (EOSFET), or a chemical field effect transistor (chemFET).

The chemical identifier 130 may also be a nanosensor. A nanosensor, for example, may be built from carbon nanotubes and have a pocket that is specific for a single type of molecule. When the target molecule enters the pocket, its presence is detected by a change in wavelength of light, a change in electrical resistance, or other suitable means. In place of carbon nanotubes, a nanosensor may be constructed from self-assembling peptides, nucleic acid nanostructures, or the like. A nanosensor may also incorporate quantum dots or other nanostructures. Other forms of nanosensors are envisioned and are within the scope of the invention.

The chemical identifier 130 may employ biosensors. A biosensor, for example, may comprise monoclonal antibodies in an automated enzyme-linked immunosorbent assay (ELISA) or similar assay. A biosensor may also utilize aptamers or other nucleic acid nanostructures to bind to target molecules, or it may use engineered proteins or peptides for this function. Adhesion of the target chemical to the sensor molecule may be measured by, for example, fluorescence (e.g. fluorescence resonance energy transfer (FRET) or fluorescence quenching), surface plasmon resonance (SPR), piezoelectric sensors, SAW sensors, quartz crystal microbalance, or the like. A biosensor may also work on electrochemical principles, producing current either by a reaction with the target chemical or from the target's natural charge or dipole using biofunctionalized ion-sensitive field-effect transistors.

The chemical identifier 130 may, for example, be a lab-on-a-chip (LOC) or other type of micro-electromechanical system (MEMS) or nano-electromechanical system (NEMS). A LOC may be combined with other sensor types and methods, depending on the target chemical. For example, detection of DNA may be effected by lysing (breaking open) cells and detecting specific DNA sequences via polymerase chain reaction (PCR), SPR, or any other suitable method known to one skilled in the art. Alternatively, detection of a target molecule by an immunoassay such as, e.g., ELISA, can be performed in a LOC. A LOC may also incorporate a DNA microarray, protein or enzyme microarray, antibody microarray, or other microarray type as may appropriate to a particular application. Many further combinations and additional techniques and methods are envisioned for a LOC-type chemical identifier (including other MEMS and NEMS identifiers) and are within the scope of the invention.

The chemical container 140 may comprise a particular chemical (such as, e.g., a toxin, a poison, a pheromone, a dye, a hormone, a drug, a pro-drug, an antigen, a peptide, a protein, a nucleic acid, a carbohydrate, a fatty acid, a signaling molecule, a neurotransmitter, or a biological waste or the like) that may be released based on the chemical release signal received from the nano RFID component 150 or the external monitoring device (not shown). The chemical may be released from the chemical container 140 through the permeable wall area (or opening) 102B of the shell 102. The chemical container 140 may have dimensions similar to or smaller than those of the nano RFID component 150. The chemical container 140 may be a passive device or a semi-passive device. In the former instance, the chemical container 140 may receive its power from the nano RFID component 150. In the latter instance, the chemical container 140 may include a power source to operate all of its functions, such as, for example, a nano battery, which may be fabricated as a nano chemical-battery or nano bio-battery, as is known in the art.

The shell 102 of the nano-sniffer 100A may include a permeable wall (or opening) 102A to allow molecules to pass into the chemical identifier 130 for detection and identification of chemicals. The shell 102 may facilitate airborne distribution of the nano-sniffer 100A to a subject or area where the subject is anticipated to come into contact with the nano-sniffer 100A. The shell 102 may facilitate intravenous injection of the nano-sniffer 100A into a subject.

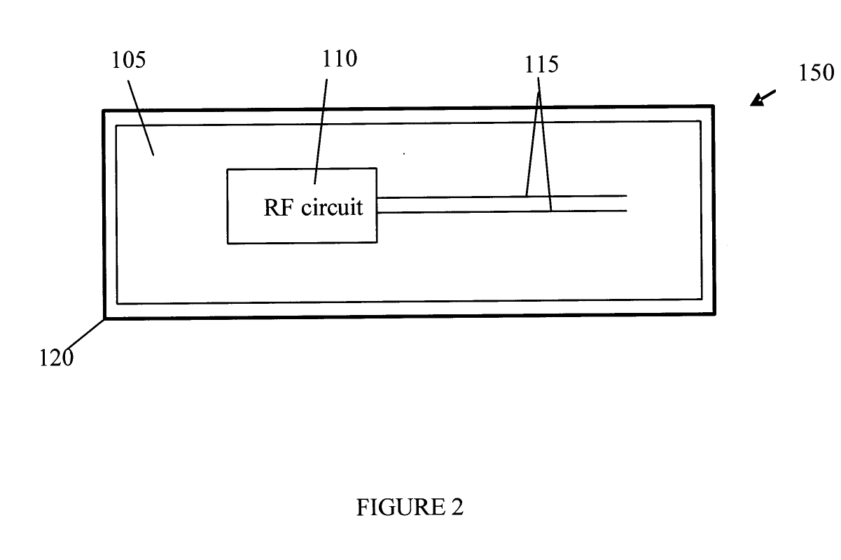

In some embodiments, the shell 102 may facilitate affixing the nano-sniffer 100A (or nano-sniffers 100B, 100C) to a particular location in a subject. The shell 102 preferably surrounds all of the circuitry and the antennae, but it may surround only the circuitry, leaving, for example, the chemical sensors or the antennae exposed. Moreover, the shell 102 may be optional, depending on intended application usage. Further, the nano RFID component of FIGS. 2-4 may be included in a plurality of vessels 100 (e.g., 100A, 100B, or 100C), each of which may be configured to detect/identify one or more chromosomes and/or release one or more chemicals. The vessels 100 may be distributed by broadcasting, which may include airborne distribution (e.g., for inhalation), contact distribution including injection/insertion, ingestion distribution (e.g., by drinking or eating), and the like.

By way of an example, the shell 102 may include nano claws (e.g., analogous to the functional properties of Velcro®) that may adhere to skin tissue, muscle tissue, blood vessels, and the like. Another example of the shell 102 may include an inorganic or organic type of adhesive (e.g., a bioglue, biological adhesives, and the like) that bonds the vessel 100 to a subject (such as, e.g., a human, animal, microorganism, inanimate object, or the like). In some applications, the shell 102 may activate adherence properties upon contact with, or in the presence of, for example, without limitation, human or animal organic properties such as skin oils, body fluids, body excretions (e.g., perspiration, saliva and the like), body proteins (e.g., hair, skin, blood, and the like). Generally, when the shell 102 is constructed to respond in some way to immediate environment characteristics, the shell 102 may be generally referred to as an environmentally reactive shell.

In other applications, the shell 102 may also be activated when the shell is in contact with a surface or material at a specific temperature range such as at human body temperature, for example, perhaps within a range of a pre-determined amount of degrees. In this way, a higher degree of success may be achieved when targeting a particular vessel 100 to a particular chromosome.

For still other applications, the shell 102 may be constructed with an adhering property that is responsive to internal body conditions such as the lungs. For example, if a subject were to inhale one or more of the distributed (perhaps by way of airborne aerosol or mist) vessels 100, the shell 102 may be activated in the presence of specific enzymes or hormones (or other compounds) present in, e.g., the lungs. Alternatively, or in addition, the shell 102 may also be constructed to respond to moisture and/or a temperature range as found in, e.g., the lungs or a blood vessel. Another example, may include when a vessel 100 is ingested, the stomach acids may activate the shell 102.

In some embodiments, the shell 102 may also be constructed with magnetic or electrostatic properties for adhering to specific types of materials, or in specific environmental conditions.

FIGS. 1B and 1C are examples of a chromosome identifier vessel 100B and a chemical delivery vessel 100C, respectively, constructed according to another embodiment of the invention.

In FIG. 1B, the chemical identifier vessel 100B includes a nano RFID component 150A and the chemical identifier 130. The nano RFID component 150A may be similar to or substantially the same as the nano RFID component 150 shown in FIG. 1A. Further, the chemical identifier 130 may be similar to or substantially the same as the chemical identifier 130 shown in FIG. 1A. The nano RFID component 150A and chemical identifier 130 may be enclosed (or contained) in a shell 107A, including a permeable wall (or opening) 102A. The shell 107A may be similar to or substantially the same as the shell 102 in FIG. 1A.

In FIG. 1C, the chemical delivery vessel 100C includes a nano RFID component 150B and the chemical container 140. The nano RFID component 150B may be similar to or substantially the same as the nano RFID component 150 shown in FIG. 1A. Further, the chemical container 140 may be similar to or substantially the same as the chemical container 140 shown in FIG. 1A. The nano RFID component 150B and chemical container 140 may be enclosed (or contained) in a shell 107B, including a permeable wall (or opening) 102B. The 107B may be similar to or substantially the same as the shell 102 in FIG. 1A.

The chemical identifier vessel 100B and the chemical delivery vessel 100C may communicate (via the nano RFID components 150A and 150B) with each other or the external monitoring device (not shown). The external monitoring device may function as a relay between the vessels 100B and 100C to provide for chemical detection and identification by the chemical identification vessel 100B and targeted chemical release by the chemical delivery vessel 100C.

The external monitoring device may be strategically located in one or more locations to activate the nano-sniffer to detect and identify a particular target and/or release a chemical that, for example, may cause the particular target to stop breathing, experience paralysis, stop the target's heart(s) from beating, experience blindness, or the like, without limitation.

FIG. 2 shows a block diagram of an embodiment of a passive nano RFID component 150. The nano RFID component 150 may include a radio frequency circuit (RF) 110 that may be configured to receive a chemical detection signal, a chemical identification signal, a chemical release signal, or the like, from any one of the chemical identifier 130, the chemical identifier vessel 100B, or the external monitoring device. Additionally, the RF circuit 110 may be configured to send a chemical detection signal, a chemical identification signal, a chemical release signal, or the like, to any one of the chemical container 140, the chemical delivery vessel 100C, or the external monitoring device. The RF circuit 110 may also receive or send the chemical detection signal, the chemical identification signal, the chemical release signal, or the like, as part of an RF transmission signal or RF reception signal, respectively. The chemical identification signal may include electronically encoded alphanumeric data to uniquely or non-uniquely identify a detected chemical.

The RF circuit 110 may be configured to receive or transmit signals when triggered by an RF excitation signal received from, for example, the external monitoring device, or the like. The RF circuit 110 may also be configured to include a memory (not shown), such as an EEROM or an EEPROM, for example, to store information regarding one or more chemical, such as a chemical type, a chemical identification number, a chemical concentration, a target chemical concentration, or the like.

The nano RFID component 150 may include antennae 115 that may receive an RF reception signal and also emit an RF transmission signal generated by the RF circuit 110. The RF transmission signal may include, for example, but is not limited to, the chemical detection signal, the chemical identification signal, the chemical release signal, or the like. The antennae 115 may be at least one, preferably two, carbon nano tubes or other nano materials suitable for RF reception (e.g., to receive the RF reception signal) and emission (e.g., to transmit the RF transmission signal, which may include an outbound backscatter signal). Also shown as part of the general nano RFID component 150 is a layer 120, such as a plastic coating or other suitable composition that provides environmental protection for the nano-RFID device 105. The nano-RFID device 105 may have a size of about 150 nanometers, or smaller, in all dimensions (length, width, and thickness).

The nano RFID component 150 may be further configured to receive the RF signal and to provide power to the chemical identifier 130 and/or the chemical container 140.

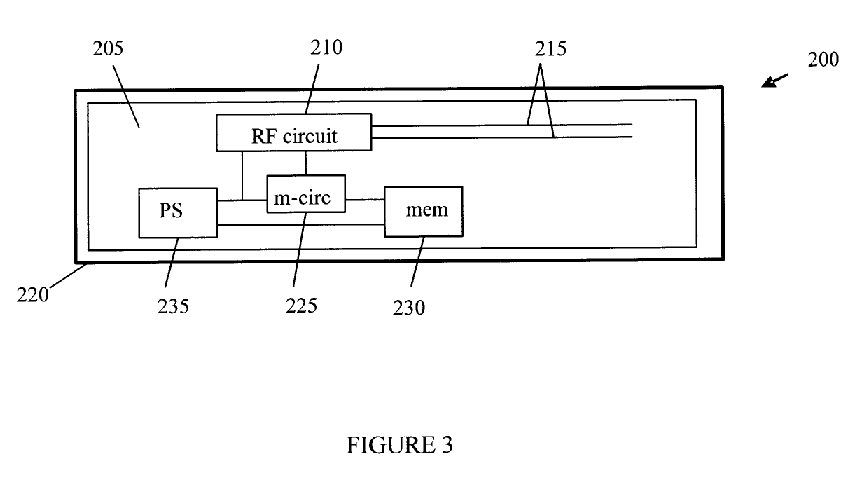

FIG. 3 is a block diagram of an embodiment of active nano RFID component, generally denoted by reference numeral 200. The nano RFID component 200 may include an active nano RFID device 205 and may include an RF circuit 210 that is configured to receive an RF reception signal and configured to send (or emit), e.g., a chemical detection signal, a chemical identification signal, a chemical release signal, or the like. The RF circuit 210 may send the chemical detection signal, the chemical identification signal (such as, e.g., electronically encoded alphanumeric data to uniquely identify the detected chemical), the chemical release signal, or the like, based on its own initiative or based on the initiative of the micro-circuit 225 (which may comprise a micro-processor, or the like) that provides additional processing and control capability. The active nano device 205 may also be configured with a memory 230, such as an EEROM or an EEPROM, for example, to store information regarding one or more chemicals, such as a chemical type, a chemical identification number, a chemical concentration, a target chemical concentration, or the like.

The active nano device 205 may also include a nano power source 235 such as a nano battery, for example. The power source 235 may be fabricated as a nano chemical-battery or nano bio-battery, as is known in the art. The power source 235 may be configured to provide power to the RF circuit 210, micro-circuit 225, and memory 230. The power source 235 may be further configured to provide power to the chemical identifier 130 and/or chemical container 140. The power source 235 may provide sufficient power to cause a stronger RF transmission signal (which may include a chemical release signal), hence greater transmission distances, as compared with a passive nano RFID device, such as shown in relation to FIG. 2, for example. Antennae 215 may receive an RF reception signal and also emit an RF transmission signal as generated by the RF circuit 210 that may be initiated by the micro-circuit 225. The antennae 215 may be at least one, preferably two, carbon nano tubes or other nano materials suitable for RF reception and/or RF transmission, including transmitting an outbound RF backscatter signal. Also, the nano RFID component 200 may include a layer 220, such as a plastic coating or other suitable composition that provides environmental protection for the nano-RFID device 205. The RF circuit 210 and the micro-circuit 225 may be combined in some embodiments. The nano device 205 may have a size of about 150 nanometers, or smaller, in all dimensions (length, width, and thickness).

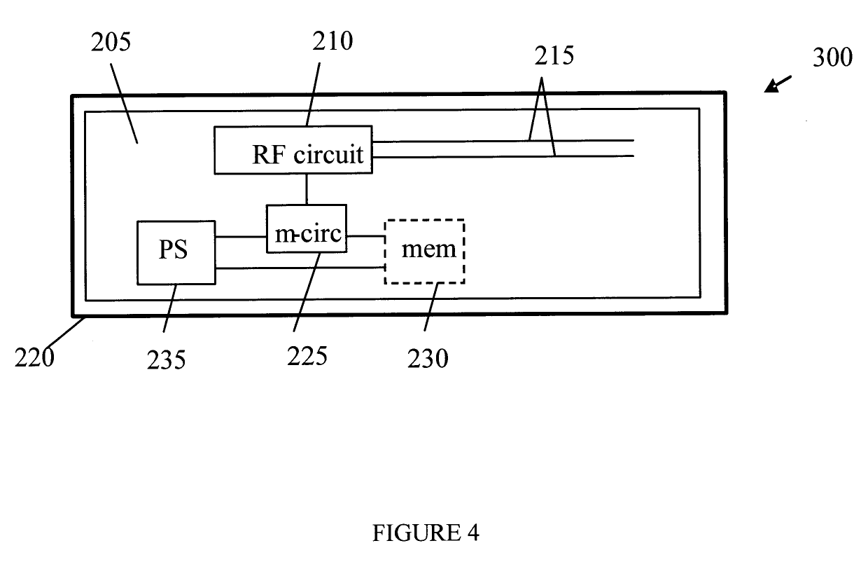

FIG. 4 is a block diagram of an embodiment of a semi-passive nano RFID component, generally denoted by reference numeral 300. The embodiment of FIG. 4 may be configured similarly to the device of FIG. 3, except that the nano power source 235 does not power the response signal. Rather, the response signal may be provided in the same manner as a passive nano RFID device (such as shown in FIG. 2, for example) by backscatter techniques. However, in some embodiments, the RF circuit 210 may be powered at least in part by the nano power source 235 for interacting with the micro-circuit 225 for exchange of information (perhaps as contained in memory 230), such as identification data, and so that the exchanged information may be transmitted (or received by micro-circuit 225), as appropriate. The nano RFID component 300 may have a size of about 150 nanometers, or smaller, in all dimensions (length, width, and thickness).

Moreover, the nano-sniffer 100 (i.e., 100A, 1008, or 100C), including, for example, the nano RFID component 150, 200, or 300, may be dynamically activated for responding to a RFID trigger query. That is, the nano-sniffer 100 may be inhibited initially when configured so that it appears to be a “dead” device, but in the presence of specific environmental triggers (e.g., a blood vessel, the lungs, stomach, proteins, fluids, compounds, temperatures, and similar environmental triggers) the device 105, 205 may change its internal state and become “active” and begin responding (by providing internal data) to external RFID triggers (i.e., when an external trigger is detected by the nano RFID device). This “dead” and subsequent “active” capability may prevent or reduce premature activation of the nano-sniffer 100 until successfully implanted into or affixed to a target, as described previously. In some embodiments, this “awakening” stimulus of a “dead” nano-sniffer 100 may be associated with or depend upon the activation of the shell 102, as described previously. That is, when the shell 102 is activated by a specific environmental condition, the vessel 100, including the nano RFID device 105, 205 may be dynamically activated and configured to respond to any subsequently detected external RFID trigger.

In some applications, the identification information within a nano RFID component 150, 200, 300 may be duplicated among more than one nano-sniffer 100 (perhaps thousands, or more, in some applications), so that more than one nano-sniffer 100 may detect the same or substantially the same chromosome and effectively deliver chemicals to various locations within (or on) the subject. The chemicals may be delivered at substantially the same time or at different times.

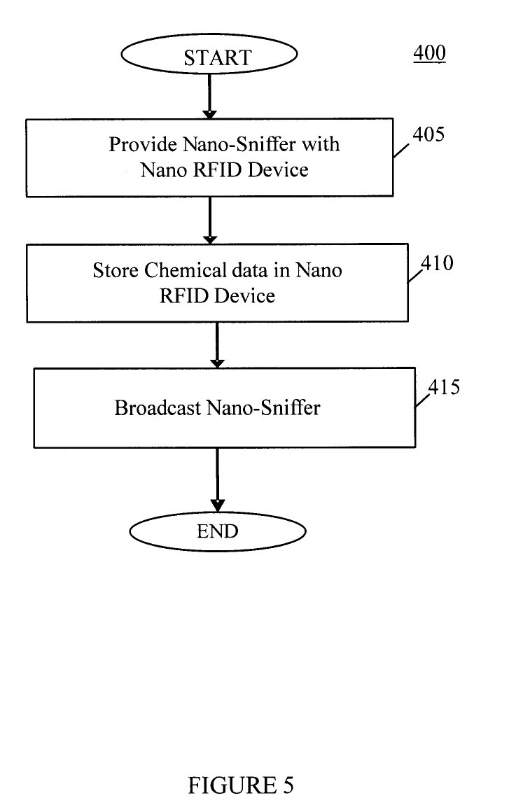

FIG. 5 is a flow diagram of an exemplary process 400 performed according to principles of the invention and programming a nano-sniffer constructed according to principles of the invention.

Referring to FIG. 5, initially, a chemical delivery vessel is provided with a nano RFID component (Step 405). Information regarding a particular chemical, such as a chemical type, a chemical identification number, a chemical concentration, a target chemical concentration, a threshold chemical concentration, or the like may be stored (Step 410). The chemical delivery vessel may then be broadcast to, for example, a subject (such as, e.g., a person, an animal, a plant, a micro-organism, an inanimate object, or the like) (Step 415). The chemical delivery vessel 100 may be broadcast by, for example, airborne dissemination or embedding the chemical delivery vessel subcutaneously into the subject (such as, e.g., injecting the chemical delivery vessel intravenously into, e.g., an artery, a vein, a capillary, or the like) or any of the other broadcasting methodologies discussed herein.

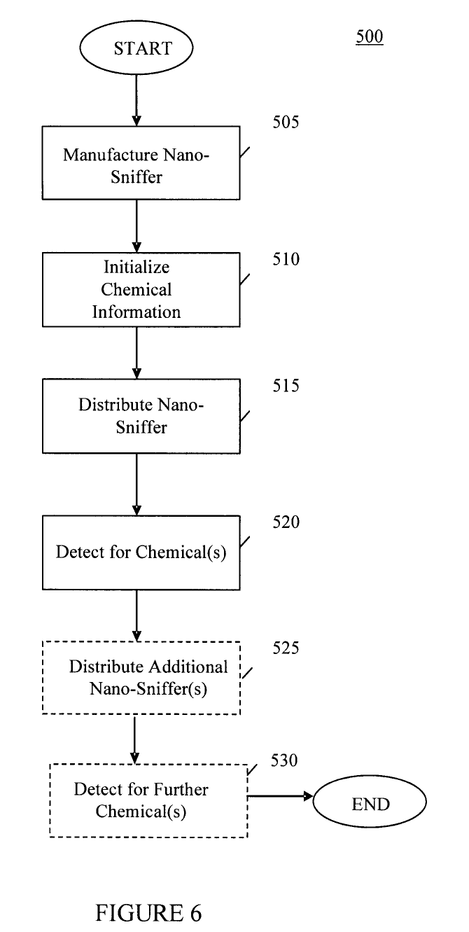

FIG. 6 shows a flow diagram showing exemplary process for constructing and distributing the nano-sniffers, according to principles of the invention.

Referring to FIG. 6, after the process 600 begins, one or more nano-sniffers may be constructed according to principles of the invention, such as described in relation to FIGS. 1A, 1B, 1C, and 2-4 (Step 505). The nano-sniffers may be constructed with any suitable shell 102, as described previously, depending on application, including an environmentally reactive shell. In some applications, no shell 102 may be needed. The one or more nano-sniffers may be initialized with chemical information suitable for an application and might include any of, for example, without limitation, a chemical type, a chemical identification number, a chemical concentration, a target chemical concentration, a date, a time, a nano-sniffer affixing location, a physical location (e.g., country or GPS coordinate), and the like (Step 510). The one or more nano-sniffers may be uniquely identified, or may have a common set of indicia. The initialized one or more nano-sniffers may be distributed, broadcasted or delivered to one or more subjects (e.g., human, animal, micro-organism, inanimate object, or the like) (Step 515). The delivery may be accomplished in nearly any suitable manner, including intravenous injection, subcutaneous injection, direct contact with or insertion into the target, or indirect delivery through a channel such as a food channel, water channel, or airborne channel and the like. One or more external monitoring devices (including, e.g., an RFID reader, an RFID transponder, or the like) may be provided at strategic private and/or public locations for triggering the nano-sniffer to activate. The external monitoring devices may be deployed at nearly any location including, for example, private or public transit points such as a home, a place of business or gatherings, airports, ships, planes, ports of entry, car rental locations, train depots, buildings, trails, and the like. Virtually any location may be equipped with an external monitoring device for activating the nano-sniffer.

Optionally, additional nano-sniffers may be distributed, perhaps having different chemical information from the first nano-sniffer (Step 525). In this manner, further chemicals may be detected and further chemicals may be strategically and timely delivered (Step 530).

As an exemplary, non-limiting application, nano-sniffers may be constructed with glucose sensors and may carry insulin or another hormone involved in regulation of blood sugar as their chemical payload. The nano-sniffers may be deployed in the blood stream, where they monitor glucose concentration. The nano-sniffers may release their payload when glucose concentration falls below or rises above a certain concentration. The nano-sniffers may be passive, measuring glucose only when powered by an RFID signal. Alternatively, the nano-sniffers may be semi-passive, monitoring glucose, e.g., at regular intervals and reporting their findings when prompted by an RFID signal.

As an additional non-limiting exemplary application, nano-sniffers may be constructed to detect pheromones, characteristic waste products, or other chemical indicators of specific pests, such as cockroaches, ants, termites, fungus, mold, rats, mice, crickets, or the like. Upon detecting the targeted pest, the nano-sniffer may release its payload, which may be a toxin designed to kill the pest, or a pheromone or other chemical designed to deter the pest from entering the area where the nano-sniffer is deployed. Nano-sniffers that sense different pests may be deployed together, such as, e.g., deploying nano-sniffers targeting cockroaches and ants to a kitchen area.

Further non-limiting examples include nano-sniffers that are deployed to the brain and detect the concentration of a neurotransmitter. In response to the concentration being above or below a threshold level, the nano-sniffer may release a neurotransmitter or pharmacological agent. Nano-sniffers may also detect antigens or other indicators of specific pathogens and release antibiotics, anti-viral drugs, or the like. Such nano-sniffers may be deployed in food packaging as an alternative or addition to traditional preservatives. Alternatively, they may be used on living creatures, including humans, by, e.g., spraying onto a wound.

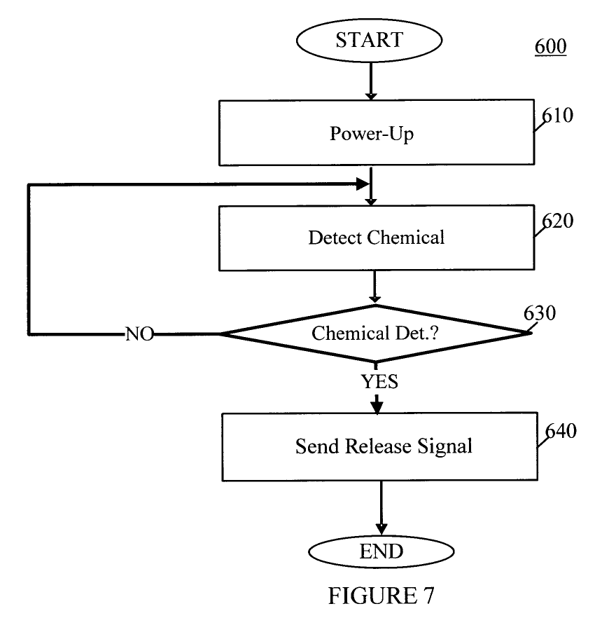

FIG. 7 shows an example of a process 600 for detecting a chemical and sending a release signal, according to principles of the invention.

Referring to FIG. 7, initially a nano-sniffer is activated (Step 610). In this regard, the nano-sniffer may be activated (powered up) based on, for example, a received RF excitation signal from an external monitoring device, or from a nano RFID component included in the nano-sniffer. The nano-sniffer may monitor for a predetermined chemical (Step 620). If the chemical is detected (YES, Step 630), then a chemical release signal may be sent to a chemical container to release a chemical contained therein (Step 640), otherwise the nano-sniffer may continue to monitor for the predetermined chemical (NO, Step 630).

It is noted that instead of detecting the predetermined chemical, the process 600 may detect whether the predetermined chemical exceeds a predetermined threshold value by a predetermined amount. For example, the process 600 may detect a blood glucose level to determine whether the level is greater than, or less than a predetermined range of values.

Relevant technology providing a foundation for enabling various techniques and principles herein may be found in several publications such as, for example: “Nanophysics and Nanotechnology: An Introduction to Modern Concepts in Nanoscience,” Edward L. Wolf, Wiley-VCH; 2 edition (Oct. 20, 2006); “Springer Handbook of Nanotechnology,” Springer, 2nd rev. and extended ed. edition (Mar. 27, 2007); “Introduction to Nanoscale Science and Technology (Nanostructure Science and Technology),” Springer, 1st edition (Jun. 30, 2004); “Fundamentals of Microfabrication: The Science of Miniaturization,” Marc J. Madou, CRC, 2 edition (Mar. 13, 2002); “RFID Essentials (Theory in Practice),” O'Reilly Media, Inc. (Jan. 19, 2006); and “RFID Applied” by Jerry Banks, David Hanny, Manuel A. Pachano, Les G. Thompson, Wiley (Mar. 30, 2007), all incorporated by reference in their entirety herein.

While the invention has been described in terms of exemplary embodiments, those skilled in the art will recognize that the invention can be practiced with modifications in the spirit and scope of the appended claims. These examples given above are merely illustrative and are not meant to be an exhaustive list of all possible designs, embodiments, applications, or modifications of the invention.

What is claimed is:

1. A nano-sniffer, including a radio frequency component, comprising:
a radio frequency (RF) component configured to be responsive to an RF signal;
a chemical identifier configured to detect a first chemical;
a chemical container configured to release a second chemical;
antennae operatively coupled to an RF section to receive the RF signal and to emit a response; and
a shell surrounding at least one of the RF component, the antennae, the chemical detector and the chemical container,
wherein the nano-sniffer is configured to be less than about 150 nanometers in at least one of width, length, and height.

2. The nano-sniffer of claim 1, wherein the shell comprises a protective covering to protect the nano-sniffer.

3. The nano-sniffer of claim 1, wherein the shell comprises an environmentally reactive layer.

4. The nano-sniffer of claim 1, wherein the shell is constructed to facilitate attaching to, or embedding in, a subject.

5. The nano-sniffer of claim 1, wherein the nano-sniffer is distributable by airborne delivery and is inhalable by at least one subject.

6. The nano-sniffer of claim 1, wherein the RF component is configured to respond by backscattering a received signal.

7. The nano-sniffer of claim 1, wherein the RF component is configured to respond with data identifying a detected chemical.

8. The nano-sniffer of claim 1, wherein the antennae comprises at least one nano carbon tube.

9. The nano-sniffer of claim 1, further comprising:
a micro-circuit to process the received signal; and
a memory operatively coupled to the micro-circuit to store chemical information.

10. The nano-sniffer of claim 1, further comprising a nano power source.

11. The nano-sniffer of claim 10, wherein the nano power source powers the RF component.

12. The nano-sniffer of claim 10, wherein the nano power source powers the RF component at least in part and the emitted response is emitted by backscatter.

13. The nano-sniffer of claim 1, wherein the RF component is dynamically configurable to be responsive or non-responsive to an RF signal based on a state of the shell.

14. The nano-sniffer of claim 1, wherein the chemical identifier comprises at least one sensor selected from the group consisting of a spectrometer, a nondispersive infrared sensor, a spectrophotometer, a potentiometric sensor, an optrode, a metal oxide semiconductor, a conducting polymer, a quartz crystal microbalance, a surface acoustic wave sensor, a microwave chemistry sensor, a chemiresistor, an electrolyte-insulator-semiconductor sensor, a metal oxide semiconductor field effect transistor, an electrolyte-oxide-semiconductor field effect transistor, and a chemical field effect transistor.

15. The nano-sniffer of claim 1, wherein the chemical identifier comprises at least one of a nanosensor or a biosensor.

16. The nano-sniffer of claim 1, wherein the chemical identifier comprises at least one of a micro-electromechanical system, a nano-electromechanical system, or a lab-on-a-chip.

17. The nano-sniffer of claim 1, wherein the first chemical comprises a toxin, a poison, a pheromone, a dye, a hormone, an antigen, a peptide, a protein, a nucleic acid, a carbohydrate, a fatty acid, a signaling molecule, a neurotransmitter, or a biological waste molecule.

18. The nano-sniffer of claim 1, wherein the second chemical comprises a toxin, a poison, a pheromone, a dye, a hormone, a drug, a pro-drug, an antibiotic, an anti-viral, a neurotransmitter, a protein, a carbohydrate, a fatty acid, a nucleic acid, a signaling molecule, a micro-electromechanical system, a nano-electromechanical system, a peptide, an aptamer, or a quantum dot.

19. A method for using a nano-sniffer, the nano-sniffer comprising:
a radio frequency (RF) component configured to be responsive to an RF signal; and
antennae operatively coupled to an RF section to receive the RF signal and to emit a response,
wherein the nano-sniffer is configured to be less than about 150 nanometers in at least one of width, length, and height,
the method comprising:
storing chemical information within the nano-sniffer; and
distributing the nano-sniffer for chemical detection and/or chemical release.

20. The method of claim 19, wherein the nano-sniffer is configured to be affixed to a human or animal subject.

21. The method of claim 19, wherein the distributing comprises airborne distributing of the nano-sniffer.

22. The method of claim 19, wherein the distributing comprises contact distribution of the nano-sniffer.

23. The method of claim 19, wherein the emitted response comprises chemical information.

24. The method of claim 19, further comprising adhering the nano-sniffer to a subject, a location, or an object.

25. The method of claim 24, wherein the adhering is achieved by an environmentally reactive shell of the nano-sniffer.

26. The method of claim 24, wherein the adhering comprises at least one of a magnetic adherence technique, an electrostatic adherence technique, and a biological adhesive.

27. The method of claim 19, wherein the distributing comprises at least one of ingestion by the subject, inhalation by the subject, or inserting into the subject.

28. The method of claim 19, wherein the nano-sniffer further comprises a shell surrounding at least the radio frequency (RF) section.

29. The method of claim 28, wherein the shell comprises at least one of an environmentally reactive shell, a shell configured for magnetic adherence, a shell configured for electrostatic adherence, or a shell configured for mechanic adherence.

30. The method of claim 28, wherein the shell comprises a protective layer.
